(19) United States
(12) Patent Application Publication
(10) Pub. No.: US 2024/0371034 A1
(43) Pub. Date: Nov. 7, 2024

(54) Heart Position Estimation

(71) Applicant: Peacs Investments B.V., Nieuwerbrug aan den Rijn (NL)

(72) Inventors: Peter Michael van Dam, Nieuwerbrug aan den Rijn (NL); Eelco Mattias van Dam, Nieuwerbrug aan den Rijn (NL); Samir Alioui, Nieuwerbrug aan den Rijn (NL)

(21) Appl. No.: 18/773,158

(22) Filed: Jul. 15, 2024

Related U.S. Application Data
(63) Continuation of application No. 17/571,783, filed on Jan. 10, 2022.
Application No. 17/571,783 is a continuation of application No. 16/651,752, filed on Mar. 27, 2020, now abandoned.

(30) Foreign Application Priority Data
Sep. 27, 2017 (NL) 2019635

(51) Int. Cl. G06T 7/73; A61B 5/00; A61B 5/25; A61B 5/318; A61B 90/98; G06K 7/10; G06K 7/14; G06T 7/00

(52) U.S. Cl. CPC G06T 7/75; A61B 5/0062; A61B 5/0064; A61B 5/0077; A61B 5/25; A61B 5/318; A61B 5/684; A61B 5/7405; A61B 90/98; G06K 7/10722; G06K 7/1413; G06K 7/1417; G06T 7/0012; G06T 7/73; G06T 2207/10024; G06T 2207/10028; G06T 2207/20021; G06T 2207/30048; G06T 2207/30168; G06T 2207/30204

(57) ABSTRACT

The present invention relates to a method to be performed by a computing device along with an ECG device for estimating an orientation of the heart based on 3-D imaging information taken from a torso, preferably applying a marker element in a process of determining an orientation of a heart, preferably in a torso model of the torso of the human body, more preferably a heart-torso model specifically including data pertaining to the heart of the, preferably human, body, the method being receiving information relating to the human body from a measurement source, such as dimensional measurement information input into the computing device from an input interface or an optical imaging device, preferably a 3D imaging device, the information being information of the exterior of the body, such as imaging information of the exterior of the torso, and determining chest dimensions, such as at the xyphoid position.

### CROSS-REFERENCE TO RELATED APPLICATIONS

This application is a continuation of U.S. patent application Ser. No. 17/571,783, filed on Jan. 10, 2022, which is a continuation of U.S. patent application Ser. No. 16/651,752, filed on Nov. 27, 2018, now abandoned, which is the United States national phase of International Application No. PCT/NL2018/000020 filed on Nov. 27, 2018, which claims priority to The Netherlands Patent Application No. 2019635 filed on Sep. 27, 2017, the disclosures of all of which are hereby incorporated herein by reference in their entirety.

### BACKGROUND OF THE INVENTION

### Field of the Invention

The present invention relates to a method to be performed by a computing device part of or coupled to an ECG device for estimating an orientation of the heart based on 3-D imaging information taken from a torso, preferably applying a marker element in a process of determining an orientation of a heart, preferably in a torso model of the torso of the human body, more preferably a heart-torso model specifically including data pertaining to the heart of the human body.

It is often preferable to have information relating to the position of the heart of a person, also when there is no scanning result available, such as a scanning result from a CT scan, an MRI scan or an echo. It may be the case that such scanning equipment is not available in a hospital or area or that there is no time to perform such scan at the moment the information relating to the position of the heart of a person is desirable, such as when performing an ECG.

As such, it is part of the inventive work of the present inventor to perceive such need and to consider the possibility of deriving a model of the torso and the heart in the torso from other means.

As such, the present invention provides a method to be performed by a computing device part of or coupled to an ECG device for estimating an orientation of the heart based on 3-D imaging information taken from a torso, preferably applying a marker element in a process of determining an orientation of a heart, preferably in a torso model of the torso of the human body, more preferably a heart-torso model specifically including data pertaining to the heart of the, preferably human, body, the method comprising steps of:
receiving information relating to the human body from a measurement source, such as dimensional measurement information input into the computing device from an input interface or an optical imaging device, preferably a 3D imaging device, the information relating to the human body comprising:
   information of the exterior of the body, at least comprising imaging information of the exterior of the torso,
determining chest dimensions, such as at the xyphoid position.

It is an advantage of such a method that the chest dimensions can be determined based on the information received from the measurement source.

According to a first preferred embodiment, the method comprises steps of estimating the heart orientation based on the chest dimensions, such as chest depth, chest width, and/or circumference. As such, preferably by applying measurements from a 3D camera, the heart position can be estimated and as such information relating to the heart position can be output by the computing device for use by for example a physician.

According to a further preferred embodiment, the steps of receiving information relating to the human body, comprise steps of receiving imaging information taken from the torso, preferably the 3D imaging information.

Further preferably, the steps of determining chest dimensions comprises steps of receiving measurement information relating to physical dimensions of the chest from input means or a database.

Preferably, the method comprises steps of determining heart dimensions of a reference model to the determined chest dimensions.

Further preferably, the method comprises steps determining a position of the xyphoid, preferably determining a virtual determination of the xyphoid point, further preferably based on at least one of the shoulder height and the length of the sternum.

Further preferably, the method comprises steps of receiving imaging information of a marker element, the marker element being arranged in an area comprising an actual position on the body, preferably on the sternum, from a marker element optical imaging device, OID, the ECG electrodes imaging device and the marker element imaging device preferably being the same imaging device,
performing an image recognition on the imaging information for obtaining a presence determination, preferably a positive or negative determination, of the marker element in the imaging information.

Preferably, the method comprises steps in which the chest depth and/or heart orientation is used in steps for estimating a torso model or a heart torso model.

Preferably, the method comprises steps in which the torso model or the heart torso model is based on measuring points such as a cloud of points based on the received information, preferably 3D imaging information.

Preferably, the method comprises steps of in which the torso model or heart torso model is based on geometrical assemblies, such as triangles, created based on the cloud of points.

Preferably, the method comprises steps of classifying partitions of the received information relating to the human body from a measurement source.

Preferably, the method comprises steps of determining at least one angle for a long axis, mitral-tricuspid axis and left basal axis.

Preferably, the method comprises steps of determining a rotation of the heart using at least one predetermined axis and angles, preferably over the three axis, preferably such that the cross section fills up about ⅓ of the thorax width.

Preferably, the method comprises steps of estimating a heart orientation based on the estimated chest dimensions.

Preferably, the method comprises steps of shifting the heart position such that the lower part of the heart coincides with the xyphoid position, preferably the heart being at a predetermined, such as minimal, distance from the chest wall.

Preferably, the method comprises steps of applying parameters when determining the position of the heart relating to the body, such as age, existing conditions, weight, fitness, chest width.

Further advantages, features and details of the present invention will be further elucidated on the basis of a description of one or more embodiments with reference to the accompanying figures.

### BRIEF DESCRIPTION OF THE DRAWINGS

FIG. 1 shows a preferred embodiment of a method according to the invention.

FIGS. 1B-1D show three embodiments of a marker element according to the present invention.

FIG. 2 shows a preferred embodiment of a further, more detailed method according to the invention.

FIG. 3 shows a preferred embodiment of a further method according to the invention.

FIG. 4 shows heart position examples relating to age and gender.

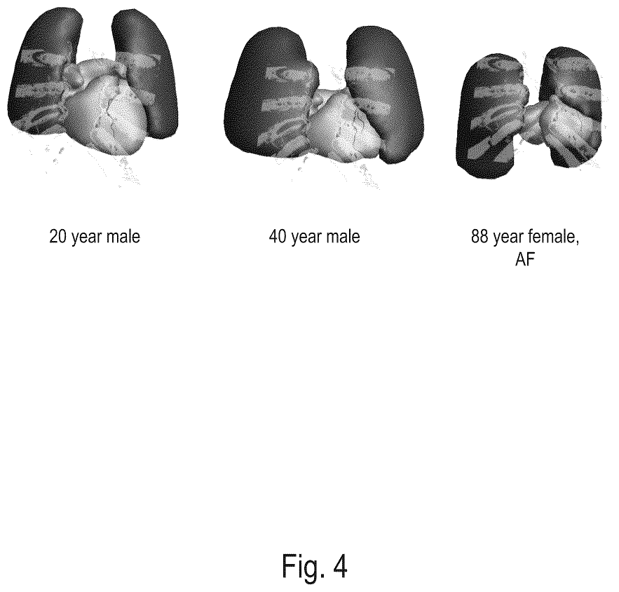

FIG. 5 shows an example of a heart in a chest and its exemplary relation to the xyphoid.

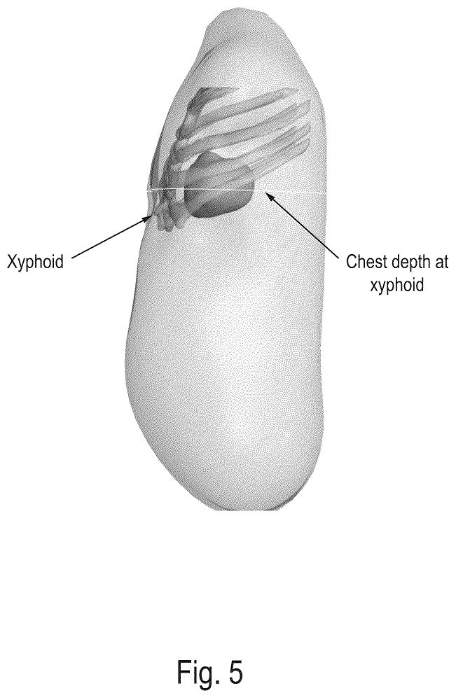

FIG. 6 shows an example of a shoulder height and sternum length in relation to a heart position in a torso.

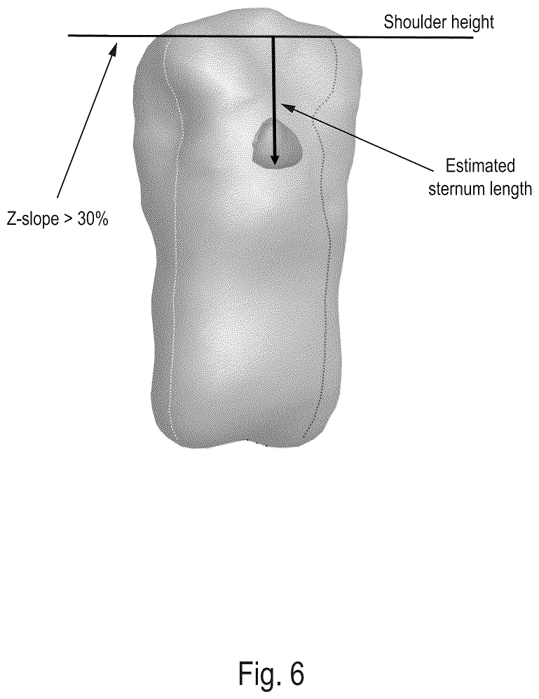

FIG. 7 Shows examples of positions in which a heart can be oriented in a torso depending on body parameters.

FIG. 8 shows an example of a heart position relative to a xyphoid or height thereof.

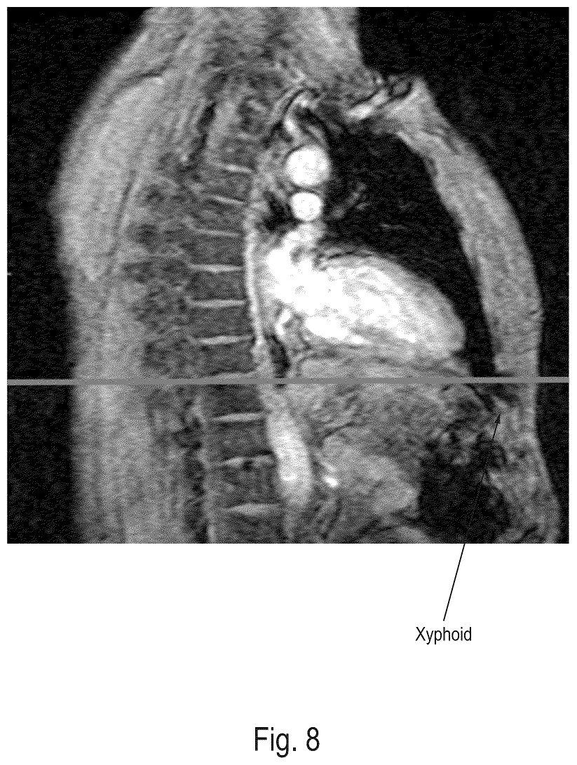

### SUMMARY OF THE INVENTION

The present invention is for instance performed in cooperation with an ECG system as input or an ECG system with features added thereto for embodying the invention. A typical embodiment comprises a computing device with receiving means for receiving from an ECG device the ECG measurements during an ECG session, such as during a procedure or for obtaining data to base a subsequent diagnosis on. The computing device is provided with a processor and memory. The memory comprises program code for enabling the processor to perform the method according to the invention.

Furthermore, the computing device is coupled to a monitor for displaying resulting images. A user interface is also displayed on the monitor for allowing input to be provided. Additional aspects of the user interface is comprised of a keyboard and mouse, touch screen, and all other user preferred in itself known input devices may be coupled to the computer through readily applicable connecting ports.

Furthermore, a 3-D camera is available for taking imaging information recordings from the torso. For obtaining the 3-D imaging information recordings, a capability to record from several sides of the torso is preferred. This is obtained by either one camera that is movable to capture images from the top, left and right side of the torso. Alternatively, two or more cameras may be fixedly mounted relative to the position of the torso in order to combine the 3-D imaging information recordings of the two or more cameras.

Furthermore, the computer is preferably connected to a database of 3-D torso models. Such a database of 3-D torso models preferably comprises unique torso models obtained by imaging devices, such as an MRI, CT or sound echo device. Depending on available time and equipment the respective information can advantageously be obtained during the ECG session, before the ECG session, or based on historical measuring data for performing of this method.

Preferably, the 3-D photo is recorded by means of a 3-D camera providing a cloud of points in a 3-D space. The cloud of points represent the subject of the imaging information recording. To this end, the 3-D camera is used to capture an image of a torso of a subject in the form of 3-D information comprising information with respect to depth and color of the subject and of the surroundings of the subject. As indicated in the above, a single camera can be moved relative to the subject, such as along a generally circular line around the torso perpendicular to a longitudinal axis of the subject. Also multiple cameras can be used mounted around the subject for taking the appropriate recordings.

When taking a photo of the torso the heart position and orientation of the heart is still unknown. Based on patient specific information a good approximation of the heart position and orientation can be made. The aortic valve is approximately behind the sternum. The lower part of the heart at the sternum is well approximated by the lower part of the sternum (xyphoid). What exactly the lower part of the heart at the sternum is depends on the orientation of the heart. So knowing the xyphoid position supports the heart position significantly.

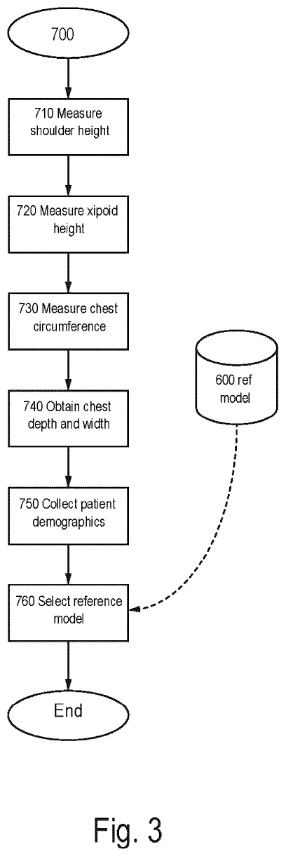

FIG. 3 provides three general examples of variations in general heart positions in a 20 year old male, in a 40 year old male and in an 88 year old female in AF view. When a heart is vertical (20 year old male example) the right heart is right, and the left ventricle is left. With increasing weight the heart is pushed upward, with means the long axis of the heart is becoming more horizontal, at the same time left chamber of the heart rotates backward. These rotations can be described as below. The chosen angles (long axis, left right angle and the left basal angle largely depends on the depth of the chest at the xyphoid.

### DESCRIPTION OF THE INVENTION

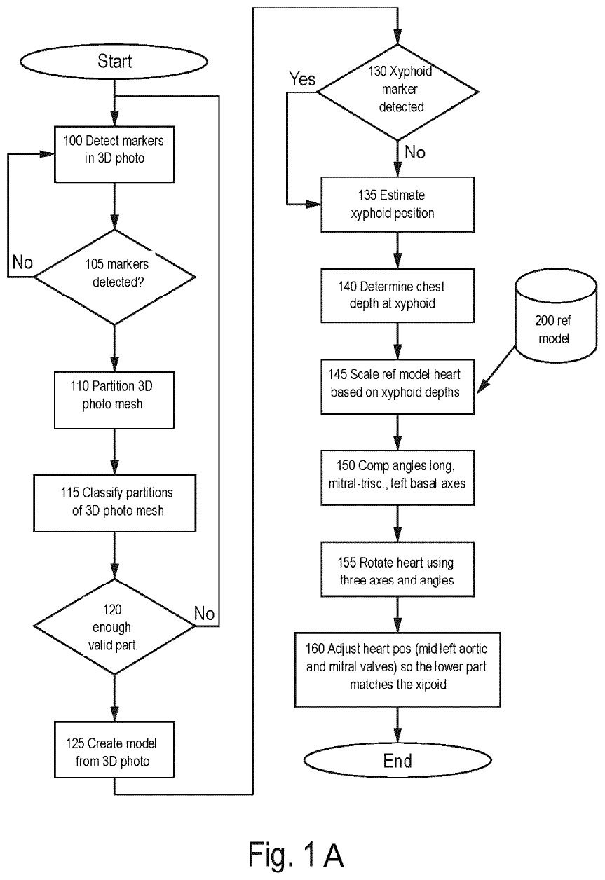

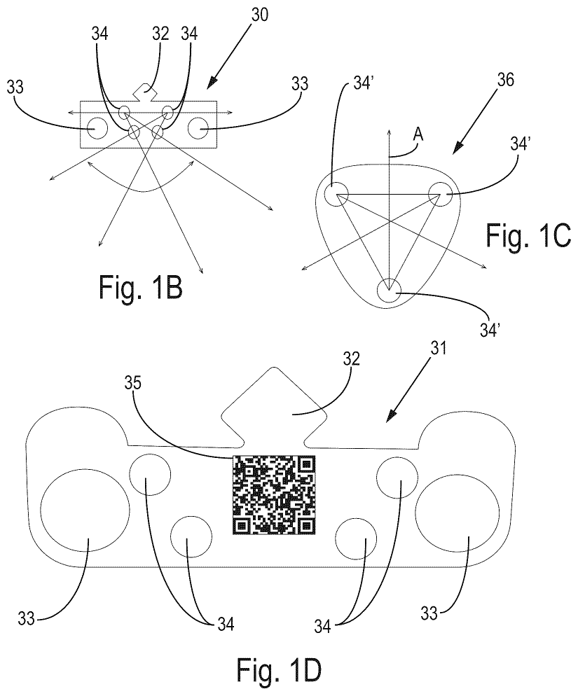

FIG. 1A shows an exemplary method as follows. Initially, optionally, a marker is detected as described relative to the below description and inserted from the priority application by reference. In step 100, markers are detected. In step 105, a step to determine whether a marker is detected is performed. In step 110, a step to partition 3D photo mesh is performed. In step 115, a step to classify partitions of 3D photo mesh is performed. In step 120, a step to determine whether there are enough valid partitions is performed. In step 125, a step to create a model from the 3D photo is performed.

Further, in step 130 the xyphoid position is determined. If a marker at this position is present this position can be used, if not determine a virtual position of the xyphoid

In step 135 virtual determination of the xyphoid point. First the shoulders height is determined from the reconstructed model. The length of the sternum is a function of the patient's length, for adults this is approximately 20 cm on average. For adults the sternum height is estimated and is used to localize the xyphoid location.

In step, a step to 140 determine the depth of the chest at the selected xyphoid position.

In step, 145 the reference model used has a defined xyphoid depth and both depths are used to scale the heart model.

In step, 150 the angles for the three axis's are determined. With less sternum depth the heart remains more vertical. When the patient is over weighted the heart is more horizontal. Axis's angles are thus determined by weight and xyphoid depth (other patient characteristics might be incorporated to tune the estimated angles.
155 a step to rotate the heart using the three estimated angles is performed.
160 a step to shift the heart position such that the lower part of the heart coincides with the xyphoid position (Z axis=vertical body axis) and is at least a minimal distance from the chest wall (x direction=xyphoid depth axis) is performed.

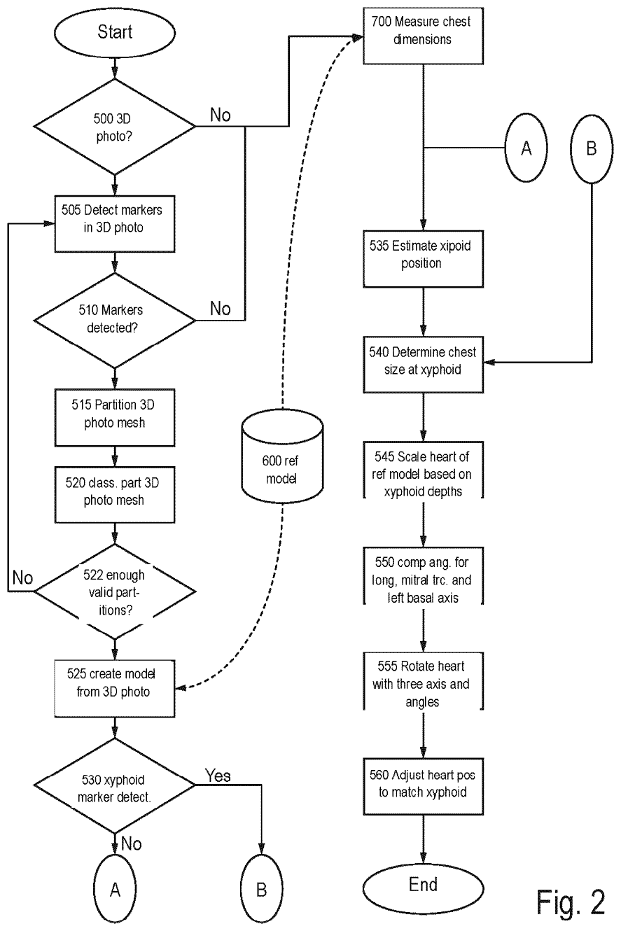

FIG. 2 shows an exemplary method as follows. In step 500, it is preferably determined if imaging information is available, i.e. 3D photo(s) from which a torso model and marker positions are to be determined. This can be any imaging from which the chest dimensions and xyphoid and/or sternum marker positions can be determined. A 3D camera is one a preferred input device.

Initially, optionally, a marker is detected as described relative to the below description and inserted from the priority application by reference in steps 505-530. In case a marker is detected, the method continues in step 540. In case no marker is detected, the method continues in step 535. Step 700 is an optional manual step if no device measurements are available to have manual measurements entered.

The database provides information as to information of models and creation of the model.

In step 530, the xyphoid position is determined. If an externally applied marker at this position is present this position can be used, if not determine a virtual position of the xyphoid based on used marker(s) or a manual indicated point on the torso. In case a Xyphoid marker is detected: the best positioning of the xyphoid is from a special marker on top of the xyphoid. With this information the sternum length can be determined as well as the lower part of the heart. Thus, such information helps with the estimation the heart position.

In step 535, virtual determination of the xyphoid point. First the shoulders height is determined from the reconstructed model. The shoulder height is determined as the position where the relative change in shoulder height along the left and right side of the body is more than a predefined slope, e.g. 30% (see figure xx). The length of the sternum is, at least preferably assumed to be a function of the patient's length, for adults this is approximately 20 cm on average. For adults the sternum height is estimated and is used to localize the xyphoid location. If no marker is present the xyphoid position can be estimated. As the sternum is in the middle of the torso, only the xyphoid z-position needs to be estimated. For adults this is on average 20 cm, decreasing with age and is slightly influenced by the thorax dimensions (depth of the sternum for instance)

Z_{xyphoid}=20−α age, the z axis is the axis from feet to head.

In step 540, the depth, width and of circumference of the chest at the selected xyphoid position is determined. Based on this also the chest ratio (chest depth divide by the chest width) can be determined. Preferably the chest dimensions at the xyphoid is determined. The width (from left and right) and depth (from front to back is calculated by the cross section of the x and y axis going through the middle of the thorax. These dimensions determine the bounding box of the heart, i.e. the ribcage.

In step 545, the reference heart model has to fit the torso model. The most limiting space is the chest depth. The depth of the heart (back to front chest direction) scaled such that this heart depth is equal to half chest depth of the model used. (The selection of the reference model should have already been described in the vector patent). Another method is to scale the heart by factor between the chest depths of the reference model and the patient specific chest dimensions having a defined xyphoid depth and both depths are used to scale the heart model. For example, when given the estimated ribcage space (540) the heart is scaled. The heart can take approximately 50% (B) of the sternum depth. The reference heart is scaled given the thorax depth at the xyphoid position:

Heart=Heart_{ref}*thorax_depth*β

In step 550, the angles for the three axes is computed. With less sternum depth the heart is relatively more vertical. When the patient is over weighted the heart is more horizontal. Axis's angles are thus determined by weight and xyphoid depth (other patient characteristics might be incorporated to tune the estimated angles. preferably, the angles for long axis, mitral-tricuspid axis and left basal axis are computed. The heart is then rotated such that is also matches the thorax width at the xyphoid position

In step 555, the heart is rotated using the three estimated angles when the chest dimensions indicate a small chest size, e.g, for instance a chest circumference of less than 1000 mm. The smaller the chest, the less space there is for the heart, the more vertical the heart will be, as is the case for children for instance the heart using the three axis and angles is rotated. Heart=rotation (Heartref, thorax_width) rotation is done over the three axis, such that the cross section fills up about ⅓ of the thorax width.

In step 560, shift the heart position such that the lower part of the heart coincides with the xyphoid position (Z axis=vertical body axis) and is at least a minimal distance from the chest wall (x direction=xyphoid depth axis) and the right side of the aortic valve are behind the sternum (y-direction). The heart position is adjusted so the lower part matches the xyphoid.

Compute the average of the points of the model aortic, pulmonary, and tricuspid valve and move this point such that this point is behind the sternum. Subsequently move the lowest part of the heart at the sternum position (the z coordinate) to z coordinate of the xyphoid point.

Heart=Heart+(x,y,z)
600 Reference torso-heart model database with models and descriptions of the models, for instance, but not limited by chest dimensions, etiology, gender, weight, height of the models, such as is a database with models of heart torso combinations.

In step 700, if no imaging data is available to determine the xyphoid and chest dimensions, e.g. chest depth at xyphoid, chest width at xyphoid height and chest circumference, the values can be measured on the patient directly with manual measurement devices. For the proper selection of the model other measurements can be taken into account here as well, such age, gender, height, weight, and etiology of the disease. Patients with a failing heart have often an enlarged left ventricle, such model should be taken from the database. Based on these measurements a model is selected from the database and scaled to match the measurements, i.e. sternum length, chest circumference and xyphoid position match the measured values.

In step 710, the shoulder height is determined, a preferred marker for this point is the manubrium of the sternum, the top of the sternum.

In step 720, the distance between shoulder height and xyphoid is determined, the lower end of the sternum.

In step 730, the chest circumference at the xyphoid position is determined.

In step 740, the chest width and depth at the xyphoid is obtained. This is preferably measured or computed using both the circumference and one of the other measures (depth or width)

In step 750, other demographics of the patient are inputted or obtained, such as weight, height, gender, etiology of a (cardiac) disease.

In step 760, based on the information collected select the reference torso-heart model that closely matches the patient specific values is determined.

A further subject of the present invention is the use of a marker element to be used as a reference point relative to the torso. According to embodiments such a marking element provides an optically recordable element, such as a surface, perform an input for the analysis of the 3-D imaging information recording. And may take the form of a patch, optionally comprising communication electronics providing an identification, having predetermined recognizable characteristics for detecting thereof by means of the computing device executing the appropriate program means. Optionally, the computing device is partly or wholly integrated into the camera device.

Preferably, the position on the thorax is predefined and enables the computing device to match, orient and or detect the thorax in the 3-D imaging information recording under clinical circumstances. By applying the marker, according to the embodiment, computing device is able to perform an analysis eliminating disturbances, such as blankets, equipment, objects or cables momentarily arranged on top of the person, etc. Also, a quality check of the 3-D photo can be based on imaging information relating to the marker element and/or the position of the camera towards the torso and/or marker.

Alternative embodiments of the marker element comprise recognition by means of color, signal, patterns, geometry, such as a shape; wherein optionally also through openings in the marker are provided for discerning the skin color.

The marker element provides a means to use as a basis for analysis. Algorithms of analysis are preferably adaptable to a range of predetermined marker types, such as distinguished by means of for example presence of information elements providing directional information such as a pattern of dots, color, shape, dimension, lighting, sound. Preferably, the position of the marker element, or marker elements on the thorax is predefined, for example by having the upper side of the marker element coincide with the upper part of the sternum or suprasternal notch and having the marker elements positioned along the sternum.

Several characteristics of several preferred embodiments of the marker element provide distinct advantages. Analysis of the color or combinations of color of the marker element provides advantages in permitting the detection of the marker element which enables analysis of an area of the subject where the marker is present. Providing a certain order of color them provide information regarding to orientations such as left, right, top, bottom and depth orientations of the subject and allow for such information to be used as inputs in the analysis.

The geometry or shape of the marker element provides the advantage of improved performance of analysis, such as during detection of the marker on the subject.

Characteristics such as sound, light or a signal from an RFID chip provided in the marker element provides advantages in the direction of the marker and advantages in performing the analysis according to the present invention, such as in identifying the marker element on the thorax of the patients.

The marker element represents a reference point towards the algorithm performing the analysis in a way that defines the 3-D space independently of how the recording of the imaging information is performed. That is, independent of which camera is used, what the orientation of the camera is, as long as the marker is comprised in the imaging information recording. The marker provides a basis for the algorithms to determine the orientation of the marker and based on that create an initial estimate of the orientation of the thorax. If non-preferable outcomes are obtained, information may be outputted as to a change in positioning of the camera are relative to the subject, such as to provide a better alignment with regard to e.g. a longitudinal axis of the torso, or to provide a better alignment relative to the marker element.

Analysis of the external shape of the subject is a further aspect in the marker element is set to improve. In case of e.g. a female subject, algorithms are provisions to detect the shape of a breast relative to the marker position. Based on this, specific analysis of the 3-D imaging information recording is performed. Advantages thereof are that the time required for performing calculations for the analysis is reduced allowing for a real-time usable results. As such, the marker element is preferably the starting point of the comparison between the 3-D imaging information recording and the 3-D model of the torso obtained.

An initial verification step in recording the 3-D imaging information recording comprises verification of the presence of the marker element. Preferably also a verification is made of acceptability of the image due to general photographic circumstances of the area or area in a room in which the recording is performed.

Furthermore, the 3-D imaging information recording is generated and verified with respect to the presence of the marker element, whereupon it is saved and used for further analysis.

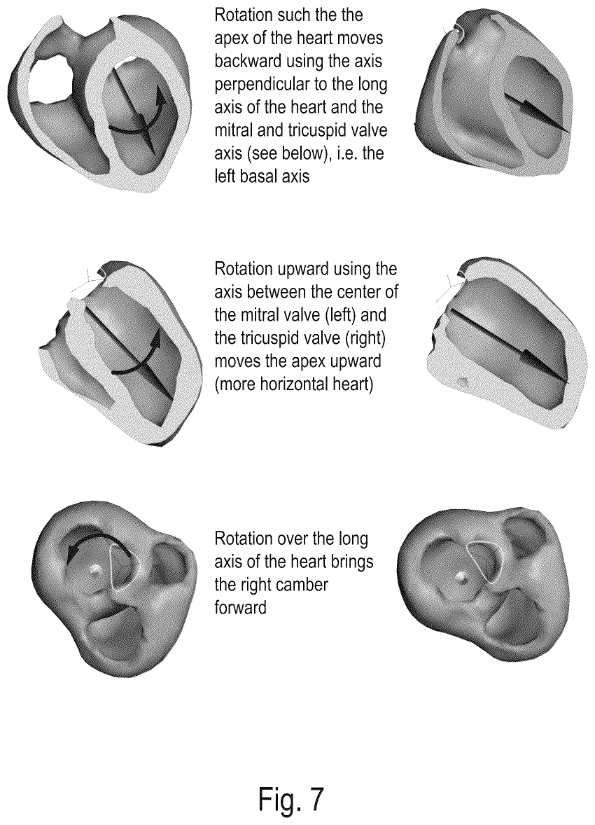

FIG. 7 generally shows examples of representations of the heart in respective views with predetermined rotations. The upper view shows a rotation such that the apex of the heart moves backward using the axis perpendicular to the long axis of the heart and the mitral and tricuspid valve axis (see below), i.e. the left basal axis. The middle view shows a rotation upward using the axis between the center of the mitral valve (left) and the tricuspid valve (right) moves the apex upward (more horizontal heart). The lower view shows a rotation over the long axis of the heart brings the right chamber forward.

The present invention is described in the foregoing on the basis of several preferred embodiments. Different aspects of different embodiments can be combined, wherein all combinations which can be made by a skilled person on the basis of this document must be included. These preferred embodiments are not limitative for the scope of protection of this document. The rights sought are defined in the appended claims.

What is claimed:

1. A method performed by a computing device part of or coupled to an ECG device for estimating an orientation of the heart based on 3-D imaging information taken from a human torso, in a process of determining an orientation of a heart of a human body, the method comprising steps of:
receiving information relating to the human body from a measurement source, via a dimensional measurement information input into the computing device from an input interface or from an optical 3D imaging device, the information relating to the human body comprising:
information of the exterior of the torso; and
determining chest dimensions,
further comprising the step of
estimating a heart orientation based on the chest dimensions, as defined by at least one of chest depth, chest width, circumference and sternum length.

2. The method according to claim 1 wherein the steps of receiving information relating to the human body comprises the step of receiving imaging information taken from the torso.

3. The method according to claim 1 wherein the step of determining chest dimensions comprises the step of determining the chest dimensions at the xyphoid position.

4. The method according to claim 1 wherein the step of determining chest dimensions further comprises the step of receiving measurement information relating to physical dimensions of the chest from input means or a database.

5. The method according to claim 1, further comprising the step of determining heart dimensions of a reference model to the determined chest dimensions.

6. The method according to claim 1, further comprising the step of determining a position of the xyphoid based on at least one of the shoulder height and the length of the sternum.

7. The method according to claim 1, further comprising the steps of receiving imaging information of a marker element, the marker element being arranged in an area comprising an actual position on the body, from a marker element optical imaging device (OID), ECG electrodes imaging device and the marker element imaging device being the same imaging device; and,
performing an image recognition on the imaging information for obtaining a presence determination, of the marker element in the imaging information.

8. The method according to claim 1 in which the chest depth and/or heart orientation is used in steps for estimating a torso model or a heart torso model.

9. The method according to claim 8 in which the torso model or the heart torso model is based on measuring points based on the received information, comprising 3D imaging information.

10. The method according to claim 1, further comprising the step of determining at least one angle for respectively at least one of a long axis, mitral-tricuspid axis and left basal axis.

11. The method according to claim 1, further comprising the step of determining a rotation of the heart using at least one of the long axis, mitral-tricuspid axis and left basal axis and respective angle, such that a cross section fills up about ⅓ of a width of the thorax.

12. The method according to claim 1, further comprising the step of shifting the heart position such that the lower part of the heart coincides with the xiphoid position, the heart being at a predetermined distance from the chest wall.

13. The method according to claim 1, further comprising the step of applying parameters when determining the position of the heart relating to the body, such as age and existing conditions.

14. The method according to claim 1, wherein the method is performed using a heart-torso model of the human body.

15. The method according to claim 1, wherein the method is performed using a heart-torso model including data pertaining to the heart of the human body.

16. The method according to claim 1, wherein the imaging information is 3D imaging information.

17. The method according to claim 6, wherein the step of determining a position of the xyphoid comprises determining a virtual determination of the xyphoid point.
